(19) United States
(12) Patent Application Publication
(10) Pub. No.: US 2010/0087444 A1
(43) Pub. Date: Apr. 8, 2010

(54) IMATINIB MESYLATE

(75) Inventors: Surya Narayana Devarakonda, Hyderabad (IN); Minakshi Asnani, Hyderabad (IN); Munaswamy Sekhar Nariyam, Hyderabad (IN); Arjun Kumar Tummala, SR., Visakhapatnam (IN); Vamsi Krishna Mudapaka, Khammam (IN); Srinivas Benda, Vijaya Nagaram (IN); Subba Reddy Peddireddy, Cuddapah (IN); Balaji Raghupati, Hyderabad (IN); Raghavendracharyulu Venkata Palle, Hyderabad (IN); Subrahmanyam Raghupati Rama Vinjamuri, Hyderabad (IN); Vijay Bhailalbhai Patel, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad, Andhra Pradesh (IN); DR. REDDY'S LABORATORIES, INC., Bridgewater, NJ (US)

(21) Appl. No.: 12/529,671

(22) PCT Filed: Mar. 12, 2008
(86) PCT No.: PCT/US2008/056588

§ 371 Date: Sep. 2, 2009

Related U.S. Application Data
(60) Provisional application No. 60/979,172, filed on Oct. 11, 2007.

(30) Foreign Application Priority Data
Mar. 12, 2007 (IN) 0497/CHE/2007
Sep. 24, 2007 (IN) 2149/CHE/2007

(51) Int. Cl. A61K 31/506; C07D 401/14

(52) U.S. Cl. 514/252.18; 544/295

(57) ABSTRACT

There is provided a solid dispersion of imatinib mesylate that includes imatinib mesylate and a pharmaceutically acceptable carrier, wherein said carrier is a cellulose derivative. Also provided is a process for making a solid dispersion and a process for making imatinib.

### TECHNICAL FIELD

The present application relates to a solid dispersion of imatinib mesylate with a pharmaceutically acceptable carrier, and processes for making it. Also provided is a process for preparation of imatinib or a salt thereof.

### BACKGROUND

Imatinib mesylate is chemically described as 4-[(4-methyl-1-piperazinyl) methyl]-N-[4-methyl-3-[[(4-(3-pyridinyl)-2-pyrimidinyl]amino]-phenyl]benzamide methane sulfonate, represented by the chemical structure of Formula (I),

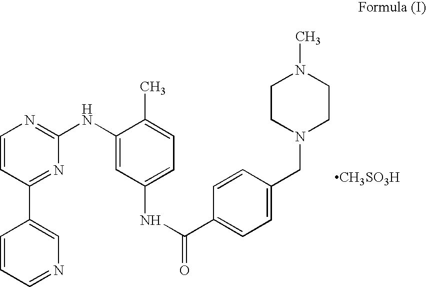

Imatinib is a protein tyrosine kinase inhibitor, especially useful in the treatment of various types of cancer and is usually administered orally in the form of methane sulfonic acid salt, i.e. in the Imatinib mesylate. Imatinib mesylate is available in the market under the brand name Gleevec in the form of tablets.

Zimmermann et al, in U.S. Pat. No. 5,521,184 disclose imatinib and the use thereof, especially as an anti-tumor agent. The '184 patent also describes a process for the preparation of Imatinib, which includes reacting N-(2-methyl-5 amino phenyl)-4-(3-pyridyl-2-pyrimidine) amine with 4-(4-methyl-piperazinomethyl)-benzoic acid chloride in the presence of pyridine as a base.

Kankan et al, in WO 2004/074502 A2 disclose a process for the preparation of imatinib that involves reacting N-(2-methyl-5-aminophenyl)-4-(3-pyridyl-2-pyrimidine) amine with 4-(4-methyl-piperazinomethyl)-benzoic acid chloride in an inert organic solvent and in the absence of external base.

Parthasaradhi et al, in U.S. Pat. No. 7,300,938 disclose crystalline imatinib mesylate Form H1, amorphous imatinib mesylate hydrate, processes for preparing them and pharmaceutical compositions.

Jegorov et al., in WO2007136510A2 disclose crystalline forms of Imatinib mesylate, amorphous form and processes for their preparation.

There remains a continuing need for the processes for preparation of imatinib and new solid forms of imatinib mesylate, which may be used for the commercial manufacturing.

### SUMMARY

In one aspect, there is provided a solid dispersion of imatinib mesylate that includes imatinib mesylate and a pharmaceutically acceptable carrier, wherein the carrier is a cellulose derivative. Various embodiments and variants are provided.

In another aspect, there is provided a process for preparing a solid dispersion of imatinib mesylate, the process including:
I. providing a solution of imatinib mesylate and a pharmaceutically acceptable carrier in a solvent, wherein the carrier is a cellulose derivative soluble in the solvent;
II. removing the solvent to obtain a residue; and
III. isolating the residue, which is the solid dispersion of imatinib mesylate.
   Various embodiments and variants are provided.

In yet another aspect, there is provided a process for preparing imatinib of Formula II or pharmaceutically acceptable salt thereof:

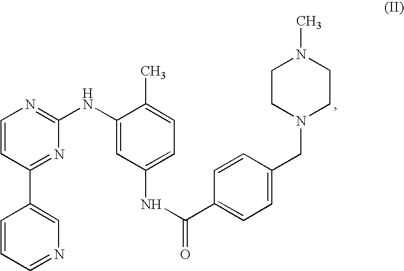

the process including reacting N-(2-methyl-5 amino phenyl)-4-(3-pyridyl-2-pyrimidine) amine of Formula IV or its salt

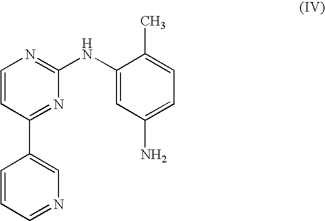

with 4-(4-methyl-piperazinomethyl)-benzoic acid of Formula III or its salt

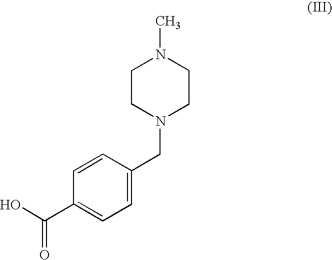

in the presence of a coupling agent. Various embodiments and variants are provided.

### BRIEF DESCRIPTION OF THE DRAWINGS

FIG. 1: Illustrative Example of X-ray powder diffraction pattern of solid dispersion prepared according to example 1.

FIG. 2: Illustrative Example of X-ray powder diffraction pattern after storage of 8 days according to example 1.

FIG. 3 Illustrative Example of X-ray powder diffraction pattern of crystalline Imatinib prepared according to example 9.

FIG. 4: Illustrative Example of differential scanning calorimetry (DSC) thermogram crystalline Imatinib prepared according to example 9.

FIG. 5: Illustrative Example of thermogram obtained after thermogravimetric analysis (TGA) of crystalline Imatinib prepared according to example 9.

### DETAILED DESCRIPTION

A single compound may give rise to a variety of solids having distinct physical properties. The variation in the physical properties frequently results in differences in bioavailability, stability, etc. between different production lots of formulated pharmaceutical products. Since polymorphic forms can vary in their physical properties, regulatory authorities require that efforts be made to identify all polymorphic forms, e.g., crystalline, amorphous, solvated, etc., of new drug substances.

Different polymorphs of drug substances generally suffer from the drawbacks of conversion to other crystalline forms on storage resulting in concomitant change, not only in the physical form and shape of the drug crystals, but also the associated dissolution and bioavailability characteristics. Generally, the molecules will revert to the thermodynamically stable form, this being the form with the least solubility. Such a thermodynamically stable form may sometimes result in a reduced or suboptimal bioavailability, especially for oral administration.

While the invention is not limited by any specific theory, it is desired to provide solid dispersion forms of drug substances, more specifically, thermodynamically stable forms of drug substances, which would have the strengths of the crystalline forms, viz. thermodynamic stability, and those of the amorphous form, viz. enhanced solubility, rapid onset of action and an enhanced bioavailability.

As used herein, the term “solid dispersion” denotes a homogeneous solid containing at least two components of different chemical identity, which components are intimately mixed with one another. Such solid dispersion is obtained when two components are present as solute in a liquid solution in a volatile solvent, and obtained as a residue upon solvent evaporation.

As set forth above, the present application provides a solid dispersion of imatinib mesylate with a cellulose derivative that serves as a pharmaceutically acceptable carrier for imatinib mesylate. While the invention is not limited by any specific theory, the solid dispersion is an intimate mixture in which the components are interspersed at a molecular level. Preferably, the solid dispersion is obtained by first obtaining a solution of the components in a volatile solvent and then removing the solvent. For example, this may be accomplished by a process, which is separately contemplated and which includes
I. providing a solution of imatinib mesylate and the cellulose derivative that is to serve as a carrier in a volatile solvent;
II. removing the volatile solvent thus generating a solid residue; and
III. isolating the residue, which is the solid dispersion of imatinib mesylate.
   While the invention is not limited by any specific theory, since the components are dissolved in the solvent at a molecular level, the residue is a solid dispersion.

It is important to differentiate between the use of cellulose derivative as a carrier, wherein it forms part of the solid dispersion, and its use as a common excipient in pharmaceutical formulation that uses a solid dispersion. In this regard, the cellulose derivative is expected to have certain properties that make it useful as a carrier. Preferably, the cellulose derivative suitable as a carrier in the solid dispersion has sufficient solubility to dissolve in the liquid solvent at levels sufficient to ensure the desired ratio of the components in the final dispersion and manufacturing suitability. Solubility in methanol may be used as a useful way to measure the desired solubility for the cellulose derivative of choice. It is preferred the suitable cellulose derivative possesses solubility in methanol equal to or greater than 0.01 g/ml, preferably, equal to or greater than 0.1 g/ml. It is also desirable for the cellulose derivative to possess a range of viscosity suitable for the final solid dispersion. The preferred viscosity of the cellulose derivative is ranging from about 1 cps to about 100 cps. In one variant, the cellulose derivative is hydroxypropylmethyl cellulose (HPMC). Preferred HPMC for use as a carrier in the solid dispersion has viscosity of 5 cps. In another variant, the cellulose derivative is ethyl cellulose.

Preferably, the solid dispersion described herein includes imatinib mesylate and the carrier present in the ratio ranging from about 5:95 to about 95:5. The more preferred ratio is about 50:50.

The inventors have discovered that the amount of residual moisture in the solid dispersion may be of importance. The residual moisture was (and may be) measured by well-accepted Karl Fisher method. Preferably, the solid dispersion contains residual moisture greater than about 1% and lesser than about 10% with respect to the weight of the solid dispersion as a whole. In one particular variant, a solid dispersion with residual moisture content less than about 2% is specifically contemplated. In another variant, which is preferred, a solid dispersion with residual moisture content ranging from about 4% to about 7% is also specifically contemplated.

Upon removal of the solvent, the residue contains imatinib mesylate in an amorphous form. Thus, a solid dispersion, in which the fraction of imatinib mesylate is present in an amorphous form is preferred and specifically contemplated. Also contemplated is solid dispersion with amorphous content ranging between 60% to 100% with respect to the weight of imatinib present in the solid dispersion, as well as dispersions with amorphous content ranging between 90% to 100%, more preferably, at about 99%.

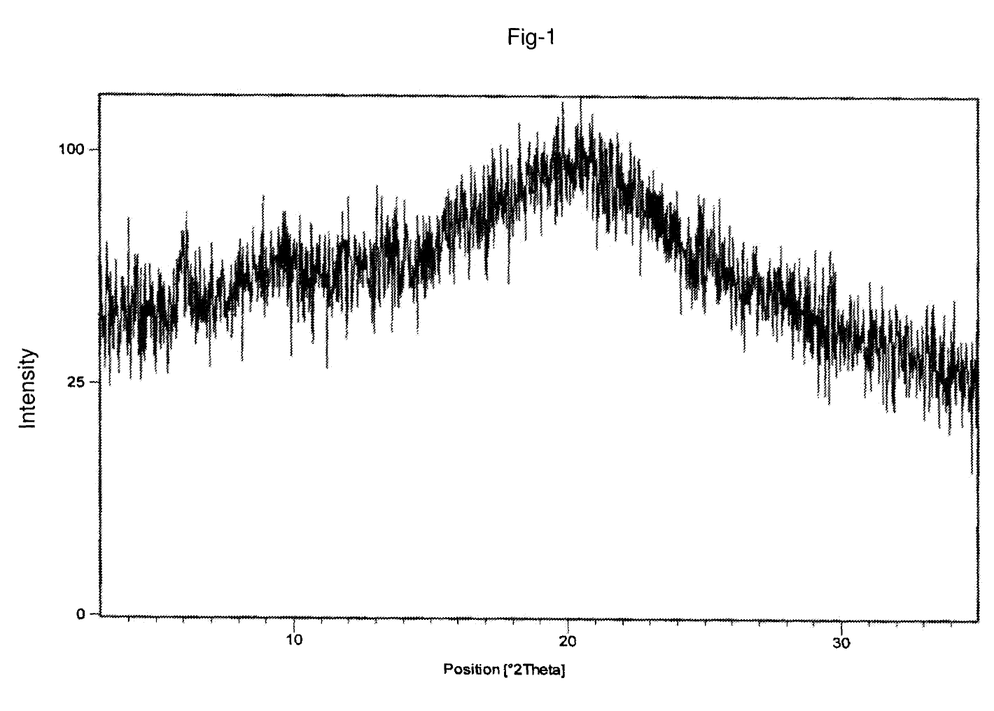

The solid dispersion described herein may be characterized by X-ray powder diffraction pattern (XRPD), Thermal techniques such as differential scanning calorimetry (DSC) and Thermogravimetric (TGA) Analysis. The samples of solid dispersion of Imatinib mesylate were analyzed by XRPD on a Bruker AXS D8 Advance Diffractometer using X-ray source—Cu Kα radiation using the wavelength 1.5418 Å. Illustrative examples of analytical data for the solid dispersion obtained in the Examples are set forth in the FIGS. 1-5 (1-2).

As set forth above, a process for making the solid dispersion is separately contemplated. Thus, there is provided a process for preparing a solid dispersion of imatinib mesylate, the process including:
I. providing a solution of imatinib mesylate and a pharmaceutically acceptable carrier in a volatile solvent, wherein the carrier is a cellulose derivative soluble in the solvent;
II. removing said volatile solvent to obtain a residue; and
III. isolating the residue, which is the solid dispersion of imatinib mesylate.

In one embodiment, the providing step includes dissolving solid imatinib mesylate and the pharmaceutically acceptable carrier in the solvent. The dissolution may be carried out at a temperature suitable for complete dissolution of the components. The starting imatinib mesylate may be of any form such as crystalline, amorphous or mixture of crystalline and amorphous forms.

In another embodiment, the providing step includes dissolving free base of imatinib in the solvent, treating the free base solution with methanesulfonic acid to obtain imatinib mesylate in situ, and adding the carrier.

The preferred volatile solvents include C1-C5 alcohols, C3-C8 esters, C2-C8 ethers, C5-C8 hydrocarbons, water, and mixtures thereof. The particular solvents suitable for the providing step include methanol, ethanol, n-propanol, isopropanol, n-butanol, isobutanol, t-butanol, water, toluene, cyclohexane, diisopropyl ether, acetone and mixtures thereof. Methanol, ethanol, n-propanol and isopropanol, water and their mixtures are preferred.

Separately contemplated are solid dispersions produced by any of the processes described herein.

Removal of solvents may be carried out by conventional methods known in the art, such as distillation, evaporation or concentration, with or without vacuum. Examples of conventional methods include, but are not limited to rotary evaporation, spray drying, freeze-drying, fluid bed drying, flash drying, spin flash drying and Ultrafilm agitated thin film dryer-vertical (ATFD-V). For example, in a typical laboratory vacuum distillation/evaporation technique, a solution is fed drop-wise into a preheated reactor under vacuum. The product is then isolated as a dry powder. It is preferred that removal of solvent is conducted with as little degradation of the dissolved components as possible.

The removal of solvent is carried out at suitable temperature related to the concentration of imatinib mesylate in the solution and the pressure under which the removal is carried out. The solvent may be evaporated under reduced pressure maintained at about 1 to 100 mbar, preferably from 10 to 30 mbar. The evaporation can be conducted at a temperature from about 30 to about 100° C. or reflux temperature.

The solid dispersion of Imatinib mesylate with the pharmaceutically acceptable carrier may isolated by any method. The process may include further drying of the product obtained with or without vacuum and in presence or absence of inert atmosphere.

The solid dispersion of imatinib mesylate with a carrier, as described herein, does not convert to any crystalline form at a temperature of about 0-5° C. or ambient temperatures at a relative humidity of less than about 50% as depicted in Table 1.

TABLE 1

| | | Sample is packed | |
|---|---|---|---|
| | | in polyethylene bag | |
| | Open Petridish & | Room temperature & | 0-5° C.& |
| Sample | 40% RH | 40% RH | 25% RH |
| Initial | Amorphous | Amorphous | Amorphous |
| After 1 day | Amorphous | Amorphous | Amorphous |
| After 7 days | Amorphous | Amorphous | Amorphous |
| After 15 days | Amorphous | Amorphous | Amorphous |

Also provided a process for preparing Imatinib of Formula II or pharmaceutically acceptable salt

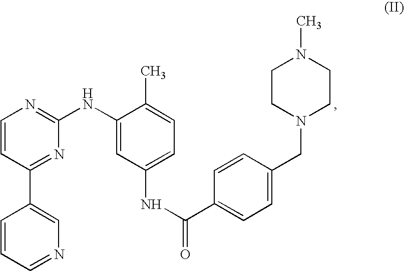

which includes reacting N-(2-methyl-5 amino phenyl)-4-(3-pyridyl-2-pyrimidine) amine of Formula IV or its salt

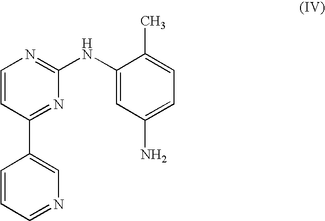

with the compound of Formula III or its salt

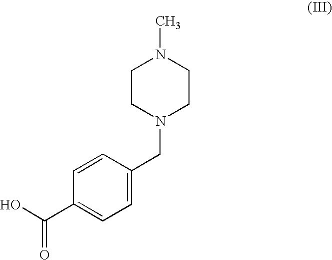

in the presence of a coupling agent to get Imatinib of Formula II or pharmaceutically acceptable salts. The use of activating agent (in addition to the coupling agent) is also contemplated.

Compound of Formula III and IV can be converted into salts, by using acids that include, but are not limited to: inorganic acids such as hydrochloric acid, hydrobromic acid, and hydroiodic acid; and organic acids such as acetic acid, tartaric acid, oxalic acid, and methanesulfonic acid.

In a preferred embodiment, there is provided a process for preparing Imatinib or pharmaceutically acceptable salts, which comprises reacting N-(2-methyl-5-aminophenyl)-4-(3-pyridyl-2-pyrimidine) amine of Formula IV with dihydrochioride salt of 4-(4-methyl-piperazinomethyl)-benzoic acid of Formula III in the presence of a coupling agent.

Non-limiting examples of suitable coupling agents include Dicyclohexylcarbodiimide (DCC), Isobutyl chloroformate, 2-chloro-4,6-dimethoxy-1,3,5-triazine (CDMT), ethyldimethyl aminopropylcarbodiimide and 2-chloro-1,3-dimethylimidazolium chloride(DMC), and mixtures thereof. Activating agents may be selected from hydroxybenzotriazole (HOBt), N-Hydroxy succinimide, and N-hydroxy piperidine.

The molar ratio of the compound of Formula IV and dihydrochloirde salt of Formula III that can be utilized in the reaction may be in the range of about 1:1 to about 1:2, preferably 1:1.5. The molar ratio of the compound of Formula IV and coupling agent that can be utilized in the reaction may be in the range of about 1:1 to about 1:2 preferably 1:1.5. The molar ratio of the compound of Formula IV and activating agent, that can be utilized in the reaction may be in the range of about 1:1 to about 1:2.5.

The reaction may be conducted in the presence of a base. Suitable bases that can be used in the process of the present application include, but are not limited to, alkali metal hydroxides such as sodium hydroxide, potassium hydroxide, lithium hydroxide; alkali metal carbonates such as sodium carbonate, sodium bicarbonate, potassium carbonate, potassium bicarbonate, lithium carbonate; amines such as triethyl amine, and trimethyl amine; N-methyl morpholine; and mixtures thereof. Preferably the base is selected from triethylamine and N-methyl morpholine.

The reaction may be conducted at a temperature of about −10° C. to about reflux temperature of the solvent. Preferably the temperature can be from about 0° C. to about 50° C.

The solvents that can be used for the said reaction include, but are not limited to, N,N-dimethyl formamide (DMF), dimethylsulfoxide (DMSO), Acetonitrile, Tetrahydrofuran (THF), Sulfolane, N-Methyl pyrrolidone (NMP), Hexamethylphosphoramide (HMPA) and dimethylacetamide (DMA) and their mixtures.

After the completion of the reaction, the product can be isolated. For instance, the reaction mixture may be quenched with water and the resulting mixture is filtered to remove the byproducts. The compound may then be isolated by extraction of the reaction mixture with a suitable organic solvent. Organic solvents that can be used for extraction of the product include but are not limited to halogenated solvents such as dichloromethane, dichloroethane, and chloroform; hydrocarbon solvents such as n-hexane, n-heptane, toluene, xylene and the like; ester solvents such ethyl acetate, butyl acetate; ether solvents such diisopropyl ether, dibutyl ether, alcohol solvents such as n-butnaol and isobutanol, ketone solvents such methyl ethyl ketone, methyl isobutyl ketone; and mixtures thereof.

The Imatinib free base thus obtained may be further purified by methods such as precipitation, crystallization or slurrying in a solvent. Solvents that may be used for such purposes include, but are not limited to esters such as ethyl acetate, n-propylacetate, isopropyl acetate and the like; ethers such as 1,4-dioxane, diethyl ether, tetrahydrofuran, diisopropyl ether, methyl tertiary-butyl ether, and the like; alcohols such as methanol, isopropanol, ethanol and the like; ketones such as acetone, ethyl methyl ketone and the like; hydrocarbons such as n-hexane, toluene, xylenes, chlorobenzene and the like; acetonitrile, water and mixtures thereof.

The solid product is recovered by suitable techniques such as decantation, filtration by gravity or by suction, centrifugation, and the like. Other techniques for separating the solids from the reaction mixtures are also within the scope of this invention.

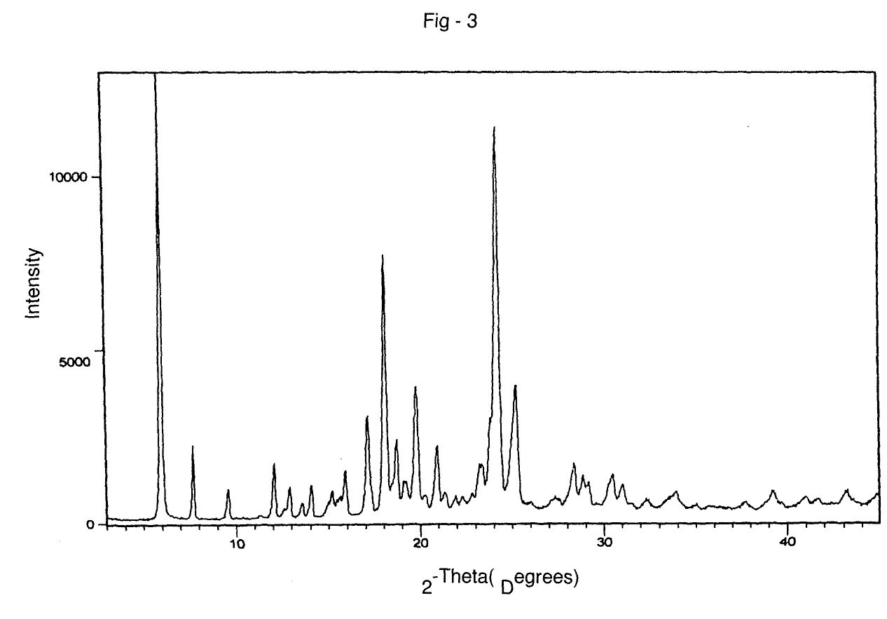

Imatinib free base obtained by the process of present application is characterized by its X-ray powder diffraction (“XRPD”) pattern, differential scanning calorimetry (“DSC”) curve, and thermogravimetric curve (TGA). Imatinib free base has characteristic peaks at diffraction angles 2-theta of about 6.0, 17.2, 18.1, 19.8, 24.3, and 25.3,±0.2 degrees. In addition to the characteristic peaks described herein above, the X-ray powder diffraction pattern may also include peaks at 7.7, 12.1, 18.7, 20.9, 23.4, and 23.8±0.2 degrees. It should be kept in mind that XRPD patterns for the same solid form typically vary as a function of a number of relevant factors, some of which include X-ray diffraction equipment and operator-to-operator variability. FIG. 3 provides an example of the X-ray powder diffraction pattern of the crystalline Imatinib free base of the present application.

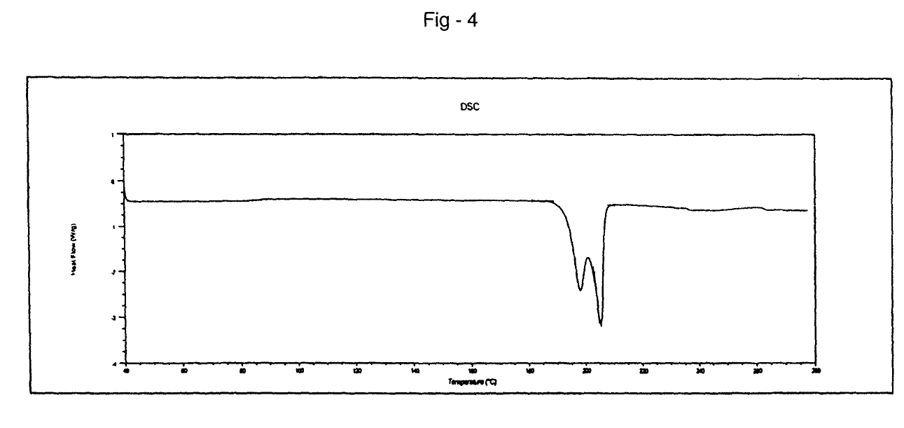

Imatinib free base obtained as per the present application has an endothermic peak at about 205.39° C. in differential scanning calorimetric (DSC) thermogram in accordance with FIG. 4.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

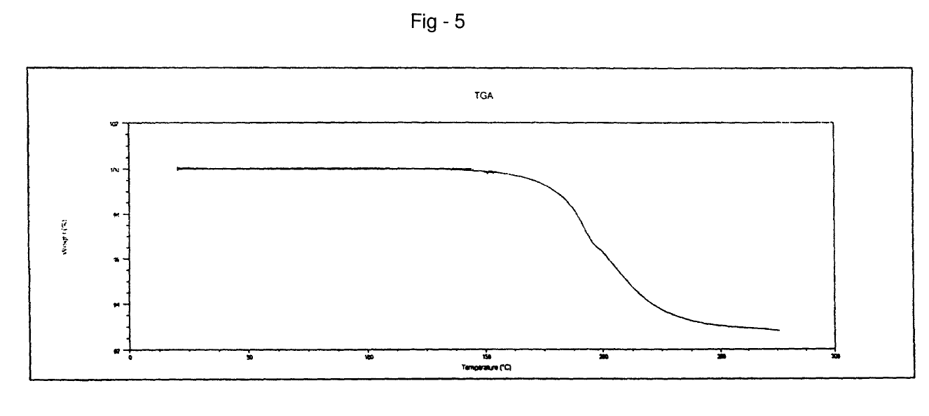

Imatinib free base of the present application has a characteristic TGA curve corresponding to a weight loss of about 0.16% w/w in accordance with FIG. 5.

Imatinib free base thus obtained can be converted into a desired pharmaceutically acceptable acid addition salt by conventional methods by reacting with a pharmaceutically acceptable acid.

Pharmaceutically acceptable acids that can be used for preparing the salt of Imatinib includes, but are not limited to: inorganic acids such as hydrochloric acid, hydrobromic acid, and hydroiodic acid; and organic acids such as acetic acid, tartaric acid, oxalic acid, methanesulfonic acid and the like.

In another embodiment, the solid dispersion obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising solid dispersion of Imatinib mesylate of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of solid dispersion of Imatinib mesylate of the present application may also include the Pharmaceutically acceptable carrier used for the preparation of solid dispersion.

Certain specific aspects and embodiments of the present application will be explained in more detail with reference to the following examples, which are provided by way of illustration only and should not be construed as limiting the scope of the invention in any manner.

### EXAMPLES

### Example 1

### Solid Dispersion of Imatinib Mesylate with Ethyl Cellulose (EC) (Ratio 50:50)

Ethyl cellulose (6 g; Fine grade—7 cps) and Imatinib mesylate (6 g; β-form) were dissolved in methanol (200 ml) while heating the flask up to 60° C. with simultaneous stirring. The resulting solution was evaporated under vacuum until dryness at a temperature of 60° C. to obtain 11 g of title compound in amorphous form (FIG. 1).

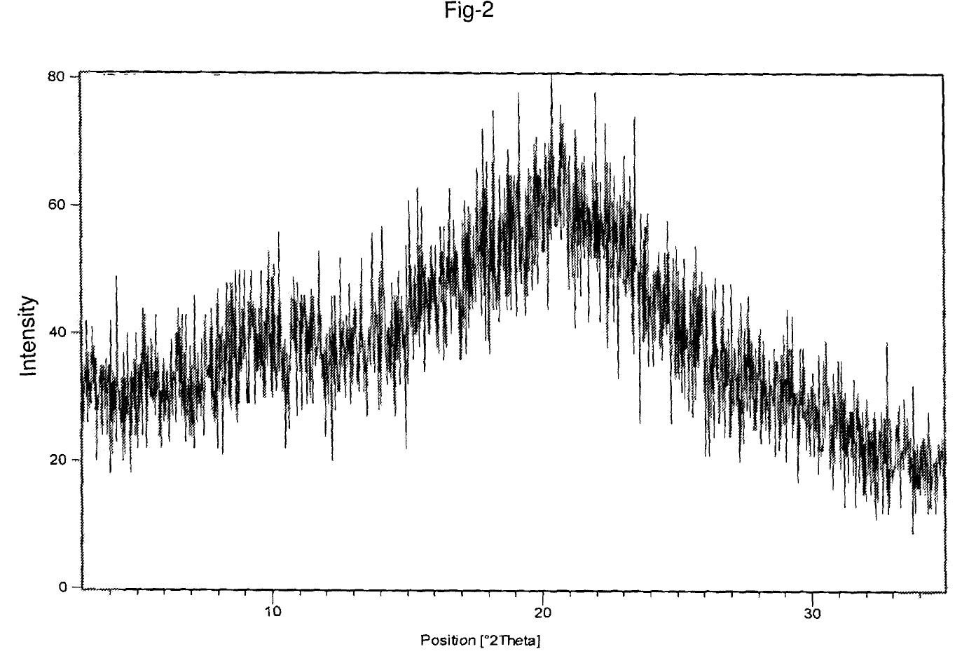

The obtained sample was exposed to different temperatures for a period of 7 days (FIG. 2) to ascertain their physical stability. The results are summarized in Table 2.

TABLE 2

| | | | Room temperature | | 0 to 5° C. (Sample | |
|---|---|---|---|---|---|---|
| | | | (Sample was packed | | was packed in poly | |
| | Open Petridish | | in poly ethylene bag) | | ethylene bag) | |
| Sample | M.C (%) | XRPD | M.C (%) | XRPD | M.C (%) | XRPD |
| Initial | 4.54 | Amorphous | 4.54 | Amorphous | 4.54 | Amorphous |
| sample | | | | | | |
| After 24 | 6.07 | Amorphous | 6.07 | Amorphous | 4.42 | Amorphous |
| hours | | | | | | |
| After 3^{rd} | — | Amorphous | — | Amorphous | — | Amorphous |
| day | | | | | | |
| After 8^{th} | — | Amorphous | — | Amorphous | — | Amorphous |
| day | | | | | | |

### Example 2

### Solid Dispersion of Imatinib Mesylate in Combination with Ethyl Cellulose (67:33)

Ethyl Cellulose (1 g; Fine grade—7 cps) was dissolved in methanol (50 ml) while heating the flask to a temperature of 60° C. Imatinib mesylate (2.0 g; β-form) was added to the obtained solution. The whole solution was evaporated completely at 60° C. under vacuum to obtain the title compound.

Yield: 2.5 g

Moisture content by Karl fisher: 1.70% w/w

### Example 3

### Solid Dispersion of Imatinib Mesylate with HPMC (Ratio 50:50)

Imatinib mesylate (6 g; β-form) and HPMC (6 g; grade—5 cps) were dissolved in methanol (410 ml) while heating the flask to a temperature of 60° C. with simultaneous stirring. The resulting solution was evaporated completely until dryness at a temperature of 60° C. under vacuum to obtain 12 g of title compound in amorphous form.

The obtained sample was exposed to different temperatures for a period of 7 days to ascertain their physical stability. The results are summarized in Table 3.

TABLE 3

| | | | Room temperature | | 0 to 5° C. | |
|---|---|---|---|---|---|---|
| | | | (Sample was packed in | | (Sample was packed | |
| | Open petridish | | poly ethylene bag) | | in poly ethylene bag) | |
| Sample | M.C (%) | XRPD | M.C (%) | XRPD | M.C (%) | XRPD |
| Initial | 6.89 | Amorphous | 6.89 | Amorphous | 6.89 | Amorphous |
| sample | | | | | | |
| After 24 | 6.78 | Amorphous | 3.41 | Amorphous | 2.63 | Amorphous |
| hours | | | | | | |
| After 2 | — | Amorphous | — | Amorphous | — | Amorphous |
| days | | | | | | |
| After 8^{th} | 3.40 | Amorphous | 4.04 | Amorphous | 4.47 | Amorphous |
| day | | | | | | |

### Example 4

### Solid Dispersion of Imatinib Mesylate with HPMC (Ratio 77:23)

Imatinib mesylate (1 g; β-form) and HPMC (0.3 g; grade—5 cps) were dissolved in methanol (20 ml) while heating the flask to a temperature of 60° C. with simultaneous stirring. The resulting solution was evaporated completely until dryness at a temperature of 60° C. under vacuum to obtain 0.807 g of title compound in amorphous form. Water content 2.81% w/w

### Example 5

### Solid Dispersion of Imatinib Mesylate with HPMC (Ratio 91:09)

Imatinib mesylate (1 g; β-form) and HPMC (0.1 g; grade—5 cps) were dissolved in methanol (20 ml) while heating the flask to a temperature of 60° C. with simultaneous stirring. The resulting solution was evaporated completely until dryness at a temperature of 60° C. under vacuum to obtain 0.750 g of title compound in amorphous form. Water content 4.24% w/w

### Example 6

### Solid Dispersion of Imatinib Mesylate without Isolating Imatinib Mesylate

Imatinib (2 g) was dissolved in methanol (210 ml) followed by addition of methane sulfonic acid (0.4 g) into it at a temperature between 25 and 35° C. and stirred the whole solution for 10 minutes. HPMC (0.4 g ; grade—5 cps) was added to the obtained reaction solution while heating the flask to a temperature of 60° C. The resultant solution was evaporated completely using spray drier to obtain 2.5 g of amorphous solid dispersion of Imatinib mesylate. M.C: 4.4% by Karl Fisher method.

Spray Drier Parameters:
Aspirator: 70%
Feed rate: 20%
Inlet Temperature: 75° C.
N₂ Pressure: 5.0 kg/cm²

The obtained sample was packed in polyethylene bag exposed to a room temperature for a period of 2 days to check the physical stability. The material was found to retain its polymorphic form after two days of holding, as indicated by maintenance of the original XRPD pattern.

Another sample was kept in a open petridish and exposed it to a room temperature for a period of 22 hours to check the physical stability. The material was found to retain its polymorphic form after two days of holding, as indicated by maintenance of the original XRPD pattern and M.C. 4.36%.

### Example 7

### Solid Dispersion of Imatinib Mesylate without Isolating Imatinib Mesylate

Imatinib (1 g) was dissolved in methanol (150 ml) followed by addition of methane sulfonic acid (0.2 g) into it at a temperature between 25 and 35° C. and stirred the whole solution for 5 minutes. HPMC (1 g; grade—5 cps) was added to the obtained reaction solution at a temperature of 30° C. The resultant solution was evaporated completely in rota-evaporator at a temperature of 65° C. and dried the solid for 30 minutes to obtain 1.2 g of amorphous solid dispersion of Imatinib mesylate.

M.C: 2.6% by Karl Fisher method

The obtained sample was packed in polyethylene bag and exposed to a temperature between 25 and 35° C. for a period of 43 hours to ascertain its physical stability. The material was found to retain its polymorphic form after 43 hours of holding, as indicated by maintenance of the original XRPD pattern and M.C.3.29%.

### Example 8

### Preparation of Amorphous Imatinib Mesylate from Imatinib Free Base

Imatinib (5 g) was dissolved in a mixture of demineralized water (20 ml) and isopropyl alcohol (175 ml) followed by addition of methane sulfonic acid (1 g) into it at a temperature between 25 and 35° C. The resultant solution was evaporated completely until dryness using spray drier to obtain 4.0 g of amorphous Imatinib mesylate.

M.C: 2.06% by Karl Fisher method.

### Example 9

### Preparation of Imatinib Free Base using Dicyclohexyl Carbodiimide as Coupling Agent and HOBt as an Activating Agent

4-(4-methyl-piperazinomethyl)-benzoic acid dihydrochloride (8.308 g), dimethylformamide (75 ml) and triethylamine (14 ml) were charged into a flask. The reaction mixture was stirred for 10 minutes at a temperature of 25° C. and then was cooled to 0° C. To the reaction mixture, hydroxybenzotriazole (HOBT) (3.657 g) and dicyclohexylcarbodiimide (DCC) (5.584 g) were added. N-(2-methyl-5 amino phenyl)-4-(3-pyridyl-2-pyrimidine) amine (5 g) was added to the reaction mixture under a nitrogen atmosphere and then the temperature was allowed to rise to 26° C. followed by stirring for 21 hours. Reaction completion was confirmed using thin layer chromatography (TLC) and then the reaction mixture was filtered. Dichloromethane (100 ml) and water (100 ml) were charged to the filtrate, and then the dichloromethane layer was separated. The dichloromethane layer was washed with 7% aqueous NaHCO₃ solution (2×50 ml). Again, the dichloromethane layer was washed with 50 ml of water. The dichloromethane layer was dried over Na₂SO₄ and evaporated completely under vacuum of 580 mm Hg at 25° C. to obtain solid.

The solid was suspended in ethyl acetate (50 ml), stirred for 15 minutes and then the suspension was filtered. The obtained solid again was suspended in ethyl acetate (25 ml), stirred for 20 minutes followed by filtration. The solid was dried under vacuum of 580 mm Hg at a temperature of 45° C. for 1 hour to afford 7 g of the title compound.

Mass: 494.4 (M+1)

### Example 10

### Preparation of Imatinib using 2-chloro-4,6-dimethoxy 1,3,5-triazine as Coupling Agent

Dimethylformamide (10 ml), 4-(4-methyl-piperazinomethyl)-benzoic acid dihydrochloride (1.65 g), N-methyl morpholine (2.54 g) and 2-chloro-4,6-dimethoxy 1,3,5-triazine (1.26 g) were charged into a flask. The reaction mixture was stirred for 2 hours. Reaction completion was checked using thin layer chromatography (TLC) and then N-(2-methyl-5 amino phenyl)-4-(3-pyridyl-2-pyrimidine) amine (1 g) was charged to the reaction mixture. The reaction mixture was stirred for 4 hours at 25° C. and then filtered. Dichloromethane (20 ml) was charged to the filtrate and then the organic layer was washed with water (3×10 ml). The organic layer was concentrated completely under vacuum of 580 mm Hg at 54° C. until dryness to obtain residue. The residue was stripped off with diisopropyl ether (3×20 ml) and then the solid was dried to obtain Imatinib.

Yield: 900 mg

Mass: 494.3 (M+1)

### Example 11

### Preparation of Imatinib using Isobutyl Chloroformate as Coupling Agent

4-(4-methyl-piperazinomethyl)-benzoic acid dihydrochloride (1.662 g), dimethylformamide (15 ml), N-(2-methyl-5 amino phenyl)-4-(3-pyridyl-2-pyrimidine) amine (1 g) and N-methyl morpholine (2.1 ml) were charged into flask. Stirred the reaction mixture for 10 minutes at 25° C. and then cooled to 2° C. To a reaction mixture, isobutylchloroformate (0.75 ml) was added over a period of 20 minutes and then stirred for 22 hours, 35 minutes under a nitrogen atmosphere. Again, isobutylchlorofomiate 0.24 ml) was added to reaction mixture and stirred for 3 hours, 45 minutes and then filtered. To the filtrate, dichloromethane (20 ml) and water (20 ml) were charged and stirred for 5 to 10 minutes. The organic layer was separated and washed with 7% aqueous sodium bicarbonate solution (2×10 ml) followed by water (10 ml). The organic layer was dried over sodium sulfate and evaporated completely until dryness. To the residue, diisopropyl ether (20 ml) was charged and stirred for 30 minutes. The suspension was filtered and then the solid was suspended in ethyl acetate (10 ml). The obtained suspension was stirred for 25 minutes and then was filtered. Ethyl acetate slurry was repeated one more time and then the obtained solid was dried at 25° C. to obtain Imatinib.

Yield: 350 mg

Mass: 494.3 (M+1)

### Example 12

### Preparation of Imatinib using Ethyl Dimethyl Aminopropyl Carbodiimide as Coupling Agent and Hydroxy Benzotriazole as an Activating Agent

4-(4-methyl-piperazinomethyl)-benzoic acid dihydrochloride (1.661 g), dimethylformamide (15 ml) and N-methylmorpholine (2.3 ml) were charged into a flask. The reaction mixture was stirred for 10 minutes at a temperature of 25° C. and then was cooled to 0° C. To a reaction mixture, hydroxybenzotriazole (0.975 g) and ethyldimethyl aminopropyl carbodiimide (1.384 g) were added, and then the whole reaction mass was stirred for 10 minutes. N-(2-methyl-5 amino phenyl)-4-(3-pyridyl-2-pyrimidine) amine (1 g) was added to the reaction mixture under a nitrogen atmosphere and then was stirred at a temperature of 26° C. for 10 hours. The reaction mixture was filtered. To a filtrate, dichloromethane (20 ml) and water (20 ml) were added, and then stirred for 5 minutes. The organic layer was separated and washed with 7% aqueous sodium bicarbonate solution (2×10 ml) followed by water (10 ml). The organic layer was dried over sodium sulfate and then evaporated completely until dryness under a vacuum of 600 mm Hg at 45° C. The obtained residue was slurred in ethyl acetate (10 ml) for 25 minutes and filtered. The slurring step was repeated one more time and then the obtained solid was dried under a vacuum of 600 mm Hg at 25° C. for 1 hour to obtain Imatinib.

Yield: 700 mg.

Mass: 494.3 (M+1)

We claim:

1. A solid dispersion of imatinib mesylate comprising imatinib mesylate and a pharmaceutically acceptable carrier, wherein said carrier is a cellulose derivative.

2. The solid dispersion of claim 1, wherein said cellulose derivative has solubility in methanol equal to or greater than 0.01 g/ml.

3. The solid dispersion of claim 1, wherein said cellulose derivative has viscosity ranging from about 1 cps to about 100 cps.

4. The solid dispersion of claim 1, wherein said cellulose derivative is hydroxypropylmethyl cellulose.

5. The solid dispersion of claim 1, wherein said cellulose derivative is ethyl cellulose.

6. The solid dispersion of claim 1, wherein imatinib mesylate and the carrier are present in the ratio ranging from about 5:95 to about 95:5.

7. The solid dispersion of claim 6, wherein imatinib mesylate and the carrier are present in the ratio of about 50:50.

8. The solid dispersion of claim 1, which has residual moisture content greater than about 1% and lesser than about 10% with respect to the weight of the solid dispersion as a whole.

9. The solid dispersion of claim 8, wherein said residual moisture content is less than about 2%.

10. The solid dispersion of claim 8, wherein said residual moisture content is ranging from about 4% to about 7%.

11. The solid dispersion of claim 1, wherein imatinib mesylate is present in an amorphous form.

12. The solid dispersion of claim 11, wherein amorphous content is ranging between 60% to 100% with respect to the weight of imatinib present in the solid dispersion.

13. The solid dispersion of claim 12, wherein said amorphous content is ranging between 90% to 100%.

14. The solid dispersion of claim 12, wherein said amorphous content is about 99%.

15. A process for preparing a solid dispersion of imatinib mesylate, said process comprising:
I. providing a solution of imatinib mesylate and a pharmaceutically acceptable carrier in a solvent, wherein said carrier is a cellulose derivative soluble in said solvent;
II. removing said solvent to obtain a residue; and
III. isolating said residue, which is the solid dispersion of imatinib mesylate.

16. The process of claim 15, wherein said providing step comprises dissolving solid imatinib mesylate and the pharmaceutically acceptable carrier in said solvent.

17. The process of claim 15, wherein said providing step comprises dissolving free base of imatinib in said solvent, treating said free base solution with methanesulfonic acid to obtain imatinib mesylate in situ, and adding said carrier.

18. The process of claim 15, wherein said cellulose derivative has solubility in methanol equal to or greater than 0.01 g/ml.

19. The process of claim 15, wherein said cellulose derivative is hydroxypropylmethyl cellulose.

20. The process of claim 15, wherein said cellulose derivative is ethyl cellulose.

21. The process of claim 15, wherein said volatile solvent is C1-C5 alcohol, C3-C8 ester, C2-C8 ether, C5-C8 hydrocarbon, water, or a mixture thereof.

22. The process of claim 21, wherein said volatile solvent is selected from the group consisting of methanol, ethanol, n-propanol, isopropanol, n-butanol, isobutanol, t-butanol, water, toluene, cyclohexane, diisopropyl ether, acetone and mixtures thereof.

23. The process of claim 17, wherein said solvent is methanol.

24. A solid dispersion produced by the process of claim 15.

25. A solid dispersion produced by the process of claim 23.

26. A process for preparing imatinib of Formula II or pharmaceutically acceptable salt thereof: 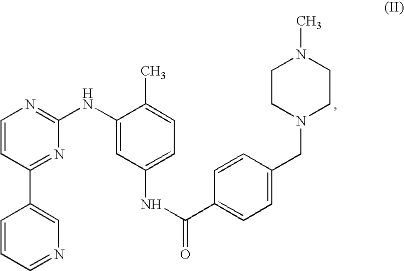
said process comprising reacting N-(2-methyl-5 amino phenyl)-4-(3-pyridyl-2-pyrimidine) amine of Formula IV or its salt 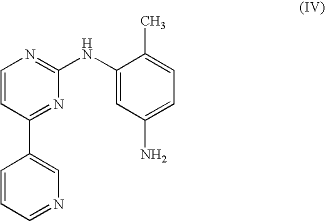
with 4-(4-methyl-piperazinomethyl)-benzoic acid of Formula III or its salt 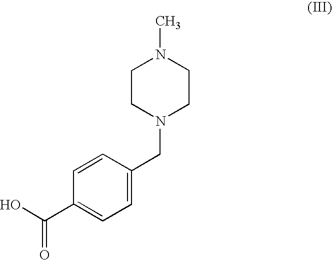
in the presence of a coupling agent.

27. The process of claim 26, wherein N-(2-methyl-5 amino phenyl)-4-(3-pyridyl-2-pyrimidine) amine is reacted with hydrochloride or dihydrochloride of 4-(4-methyl-piperazinomethyl)-benzoic acid.

28. The process of claim 27, wherein N-(2-methyl-5 amino phenyl)-4-(3-pyridyl-2-pyrimidine) amine is reacted with dihydrochloride of 4-(4-methyl-piperazinomethyl)-benzoic acid, at the molar ratio ranging from about 1:1 to about 1:2.

29. The process of claim 28, wherein N-(2-methyl-5 amino phenyl)-4-(3-pyridyl-2-pyrimidine) amine and dihydrochloride of 4-(4-methyl-piperazinomethyl)-benzoic acid are reacted at the molar ratio of about 1:1.5.

30. The process of claim 26, wherein the compound of Formula IV and coupling agent are present at molar ratio ranging from about 1:1 to about 1:2.

31. The process of claim 26, wherein the compound of Formula IV and coupling agent are present at molar ratio of about 1:1.5.

32. The process of claim 26, wherein said coupling agent is selected from group consisting of dicyclohexylcarbodiimide (DCC), isobutyl chloroformate, 2-chloro-4,6-dimethoxy-1,3,5-triazine (CDMT), ethyl dimethyl amino propyl carbodiimide, 2-chloro-1,3-dimethylimidazolium chloride(DMC), and mixtures thereof.

33. The process of claim 26, further comprising carriying out the coupling in the presence of an activating agent.

34. The process of claim 33, wherein said activating agent is selected from hydroxybenzotriazole (HOBt), N-Hydroxy succinimide, and N-hydroxy piperidine.

35. The process of claim 33, wherein the compound of Formula IV and activating agent are present at molar ratio ranging from about 1:1 to about 1:2.5

36. The process of claim 26, further comprising converting free base of imatinib into mesylate salt of imatinib.
